(19) United States
(12) Patent Application Publication
(10) Pub. No.: US 2008/0243126 A1
(43) Pub. Date: Oct. 2, 2008

(54) Rotary angled scraper for spinal disc space preparation

(76) Inventors: Robert Gutierrez, Huntington Beach, CA (US); Tyler Haskins, Oceanside, CA (US); Moti Altarac, Irvine, CA (US)

Correspondence Address: RIMAS LUKAS;VERTIFLEX, INC., 1351 CALLE AVANZADO, SAN CLEMENTE, CA, 92673, US

(21) Appl. No.: 12/079,096

(22) Filed: Mar. 25, 2008

Related U.S. Application Data
(60) Provisional application No. 60/920,218, filed on Mar. 26, 2007.

(51) Int. Cl. A61B 17/00

(52) U.S. Cl. 606/84

(57) ABSTRACT

A scraper instrument for preparing an intervertebral disc space is disclosed. The scraper instrument incorporates a uniquely configured end bit having a cutting head with a cavity for passing debris therethrough. Variations of the scraper instrument include one or more angled portions and shaft assemblies that transmit rotational torque applied at the proximal end of the instrument through the at least one angled portion and to the distally located end bit. An adjustable angled portion in addition to a bayonet-like angled portion is disclosed.

### CROSS REFERENCE TO RELATED APPLICATIONS

This application claims priority to and is a continuation-in-part of U.S. Provisional Patent Application Ser. No. 60/920,218 entitled “Rotary angled scraper for spinal disc space preparation” filed on Mar. 26, 2007, hereby incorporated by reference in its entirety.

### FIELD

The present invention generally relates to surgical instruments and methods. More particularly, but not exclusively, the present invention relates to instruments and methods for preparing the intervertebral disc space to receive an implant therebetween.

### BACKGROUND

Intervertebral spinal discs, which are located between endplates of adjacent vertebral bodies, stabilize the spine and distribute forces between the vertebrae and cushion vertebral bodies. Spinal discs may become displaced or damaged due to trauma, disease or aging. The deterioration or movement of the disc often results in the two adjacent vertebral bodies coming closer together. As a result, a deteriorated or slipped disc may produce instability of the spine, decreased mobility, nerve damage, pain and discomfort for the patient.

A common treatment is to surgically restore the proper disc space height to alleviate the neurological impact of the collapsed disc space. Sometimes, the treatment includes a discectomy in which the damaged disc is partially or completely removed. In some situations, the majority of the disc nucleus is removed leaving a majority of the disc annulus in place. The discectomy is often followed by a restoration of normal disc space height and fusion of the adjacent vertebrae to one another through the disc space. The disc space is the space previously occupied by the spinal disc interposed between the adjacent vertebral bodies.

Based on surgeon preference, access to a damaged disc space may be accomplished from several approaches to the spine following standard surgical techniques to gain access to the selected disc space and vertebral endplates. One approach is to gain access to the anterior portion of the spine through the patient's abdomen. In an anterior approach, extensive vessel retraction is often required and many vertebral levels are not readily accessible from this approach. A posterior approach may also be utilized which typically requires that both sides of the disc space on either side of the spinal cord be surgically exposed requiring a substantial incision or multiple access locations such as in posterior lumbar interbody fusion (“PLIF”) surgery. Also, a posterior lateral approach can be employed. An example of a posterior lateral approach is transforaminal lumbar interbody fusion (“TLIF”) surgery which can be performed in a minimally invasive manner. The posterior and posterior lateral approaches require a facetectomy to partially or completely remove a facet joint. Once access to the disc spaced is gained, specialized instruments are required to perform the discectomy and prepare the vertebral endplates.

In order to restore proper disc space height, one or more fusion cage, implant and/or bone graft is placed into the disc space following discectomy. The one or more fusion cage, implant and/or bone graft, for example, occupies a significant portion of the disc space to provide a large surface area over which fusion can occur. In order to promote oseointegration of the implant and fusion through the disc space, the implant is typically provided with bone graft material and the endplate surfaces of the adjacent vertebral bodies facing the disc space are prepared prior to implantation of the cage and bone graft using various procedures. In one procedure, the endplates are prepared by scraping with a scraper or rasper to expose the nucleus of the vertebral body and to promote bleeding such that a sufficient amount of blood will flow into the implant subsequently positioned between the adjacent vertebrae. Scrapping to promote blood flow at the endplates invokes the healing process of the bone, enhances bone growth and encourages more rapid and secure fusion of the implant with the adjacent vertebrae.

Furthermore, even though the implant is shaped to conform to the intervertebral disc space to provide stability and promote fusion, the implant, however, cannot always be shaped to precisely fit the complex contours of the vertebral endplates adjacent the disc space. Hence, the vertebral endplates are prepared as much as possible to match the implant in order to provide the greatest possible interface congruity between the endplates and the implant, as well as provide for the optimal contact surface, enhanced fusion area, and enhanced graft and construct stability. In order to achieve this, the amount of bone removed must be to a specified depth and width. Excess removal or penetration of the vertebral endplate can result in a weakening of the structural integrity of the vertebrae. Conversely, where an insufficient amount of bone is removed, blood flow may be very limited thereby hindering fusion of the implant to the vertebrae. Since the vertebral endplates are generally quite strong, it is desirable to preserve this structure even while skillfully removing selected portions of the bone. After the surfaces are prepared, the implant is inserted. With the implant inserted into the disc space, the vertebrae are positioned apart, more space is created for relieving impinged nerves, the positional relationship between adjacent vertebrae is restored, and graft material is introduced into the disc space to help promote the fusion process.

This invention sets forth improved instruments and methods for the effective preparation of adjacent vertebral endplates in a spinal fusion procedure.

### SUMMARY

According to one aspect of the invention, a scraper instrument having a shaft assembly with at least one angled portion is disclosed. The shaft assembly includes an outer shaft with a central bore, an inner shaft disposed inside the central bore and permitted to rotate with respect to the outer shaft, and a flexible shaft connected to the distal end of the inner shaft such that the flexible shaft is located at the angled portion.

An end bit having a cutting head with at least one cutting surface is connected to the end of the flexible shaft. The scraper instrument is configured such that rotational torque applied to the proximal end of the inner shaft is transmitted to the flexible shaft at the angled portion and to the end bit to rotate the cutting head of the end bit.

According to another aspect of the invention, a scraper instrument includes a rotatable inner shaft and a rotatable end bit having a cutting head which is connected to the inner shaft. The instrument includes an outer shaft connected to the inner shaft such that the inner shaft is permitted to move relative to the outer shaft.

According to another aspect of the invention, a method includes the step of providing a scraper instrument having a rotatable end bit with at least one cutting surface. A working channel to the disc space of a patient is created and the scraper instrument is inserted through the working channel to the disc space in a posterolateral approach that is in the range of an angle of approximately 0-90 degrees relative to an anterior-posterior axis of a patient's spine. The instrument is handled to rotate the end bit to remove tissue from the disc space, changing the angle of the instrument to pass the end bit over substantially the entire area of a vertebral endplate.

Other advantages will be apparent from the description that follows, including the drawings and claims.

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures:

FIG. 1 is a perspective view of an angled scraper instrument according to a variation of the present invention.

FIG. 2 is a top planar view of an angled scraper instrument without a handle according to a variation of the present invention.

FIG. 3 is a perspective view of a straight scraper instrument according to a variation of the present invention.

FIG. 4A is a perspective view of an end bit of a scraper instrument according to a variation of the present invention.

FIG. 4B is a top planar view of an end bit of a scraper instrument according to a variation of the present invention.

FIG. 4C is a cross-sectional view along line A-A of FIG. 4B of an end bit of a scraper instrument according to a variation of the present invention.

FIG. 4D is a side view of an end bit of a scraper instrument according to a variation of the present invention.

FIG. 4E is a cross-sectional view along line B-B of FIG. 4D of an end bit of a scraper instrument according to a variation of the present invention.

FIG. 4F is a cross-sectional view along line C-C of FIG. 4D of an end bit of a scraper instrument according to a variation of the present invention.

FIG. 4G is a cross-sectional view along line B-B of FIG. 4D of another variation of the end bit of a scraper instrument according to a variation of the present invention.

FIG. 5 is perspective view of a flexible shaft of a shaft assembly of a scraper instrument according to a variation of the present invention.

FIG. 6A is a perspective view of a coupler of a shaft assembly of a scraper instrument according to a variation of the present invention.

FIG. 6B is a cross-sectional view of a coupler of a shaft assembly of a scraper instrument according to a variation of the present invention.

FIG. 7A is a side view of an inner shaft of a shaft assembly of a scraper instrument according to variation of the present invention.

FIG. 7B is an end view of the proximal end of an inner shaft of a shaft assembly of a scraper instrument according to a variation of the present invention.

FIG. 8 is a perspective view of a ring of a shaft assembly of a scraper instrument according to a variation of the present invention.

FIG. 9A is a cross-sectional view of an outer shaft of a shaft assembly of a scraper instrument having an angled portion according to a variation of the present invention.

FIG. 9B is a cross-sectional view of an outer shaft of a shaft assembly of a scraper instrument having an angled portion according to a variation of the present invention.

FIG. 9C is a cross-sectional view of an outer shaft of a shaft assembly of a scraper instrument having two angled portions according to a variation of the present invention.

FIG. 9D is a cross-sectional view of an outer shaft of a shaft assembly of a scraper instrument having two angled portions according to a variation of the present invention.

FIG. 10 is a cross-sectional view of a scraper instrument according to a variation of the present invention.

FIG. 11A is top planar view of a handle of a scraper instrument according to a variation of the present invention.

FIG. 11B is a perspective view of a connecting portion of a handle of a scraper instrument according to a variation of the present invention.

FIG. 12A is a perspective view a scraper instrument according to a variation of the present invention inserted between two adjacent vertebral bodies.

FIG. 12B is a top view a scraper instrument according to a variation of the present invention inserted between two adjacent vertebral bodies.

### DETAILED DESCRIPTION

Before the subject devices, systems and methods are described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Unless defined otherwise, all technical and scientific terms used herein have the same meaning as commonly understood by one of ordinary skill in the art to which this invention belongs.

It must be noted that as used herein and in the appended claims, the singular forms “a”, “an”, and “the” include plural referents unless the context clearly dictates otherwise. Thus, for example, reference to “a spinal segment” may include a plurality of such spinal segments and reference to “the screw” includes reference to one or more screws and equivalents thereof known to those skilled in the art, and so forth.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

The present invention will now be described in detail by way of the following description of exemplary embodiments and variations of the systems and methods of the present invention. While more fully described in the context of the description of the subject methods of implanting the subject systems, it should be initially noted that in certain applications where the natural facet joints are compromised, inferior facets, lamina, posterior arch and spinous process of superior vertebra may be resected for purposes of implantation of certain of the dynamic stabilization systems of the present invention. In other applications, where possible, the natural facet joints, lamina and/or spinous processes are spared and left intact for implantation of other dynamic stabilization systems of the present invention.

Reference will now be made in detail to the present embodiments of the invention, examples of which are illustrated in the accompanying drawings. Wherever possible, the same reference numbers will be used throughout the drawings to refer to the same or like parts. In addition, each of the inventive embodiments described herein may be employed in a percutaneous procedure, a mini-open procedure or an open procedure. Utilization of minimally invasive techniques can shorten the procedure's time and speed recovery by the patient. However, the application of these inventions in a minimally invasive manner is not a requirement.

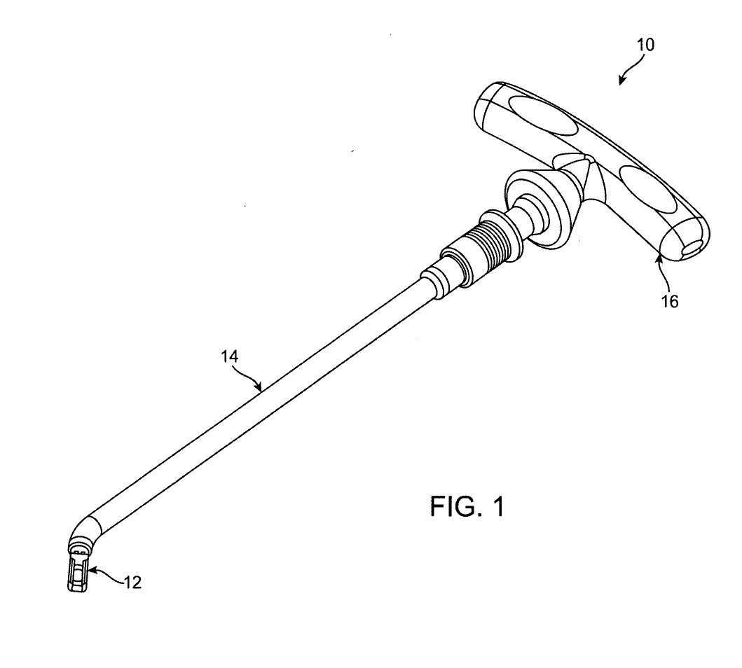

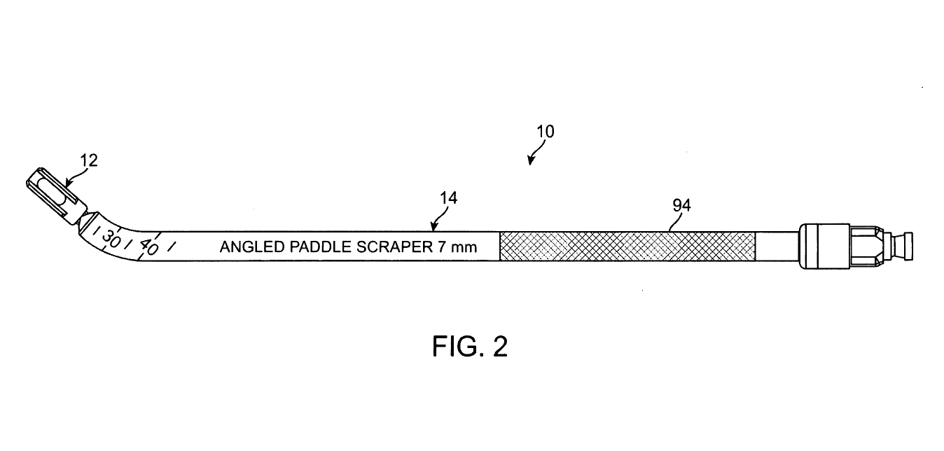

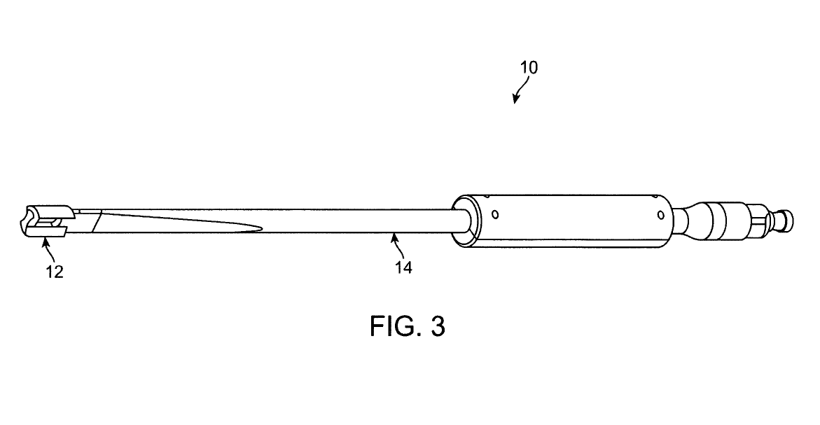

FIG. 1 illustrates a perspective view of a scraper or reamer 10 according to the present invention. The scraper 10 includes an end bit 12, a shaft assembly 14 and a handle 16. The handle 16 is connected to the shaft assembly 14 which is connected to the end bit 12. FIG. 2 illustrates a top planar view of the scraper 10 with a removable handle 16 removed. Although FIGS. 1 and 2 show an angled scraper 10, other variations of the present invention include a bayonet style scraper and a straight scraper 10. A straight scraper is shown with handle 16 removed in FIG. 3. In normal use, the scraper instrument 10 is oriented so that the handle 16 is located proximally and accessible by the surgeon and the opposite end of the instrument 10 is oriented distally away from the surgeon and towards the operative site. The operative site is generally the spinal column of a patient and, in particular, the disc space and adjacent vertebral bodies. The length of the instrument allows the surgeon to reach intended areas of modification and at the same time protect surrounding soft tissue structures including the spinal cord and nerves.

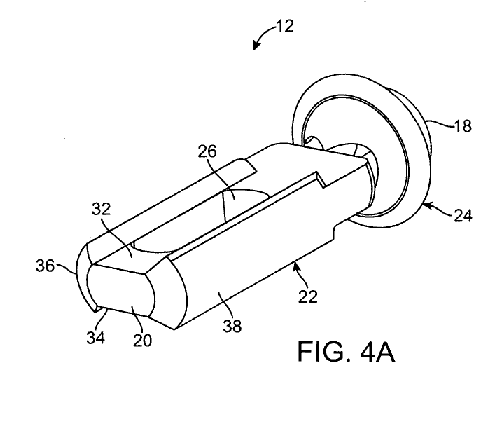

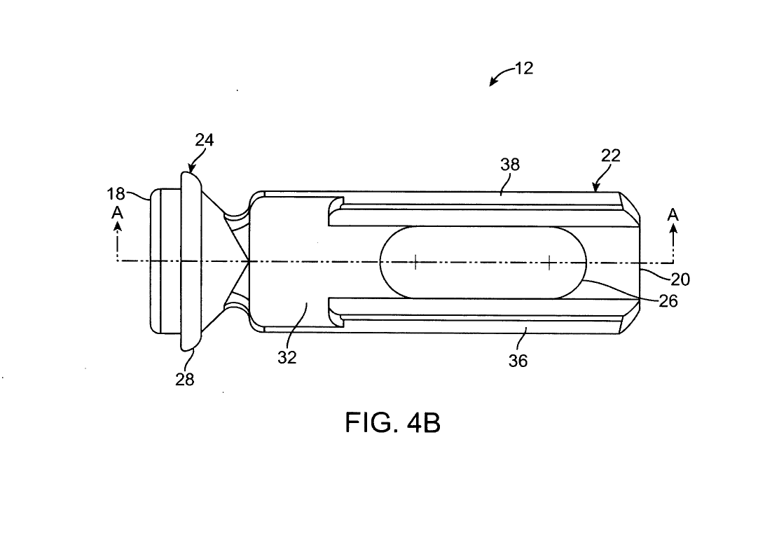

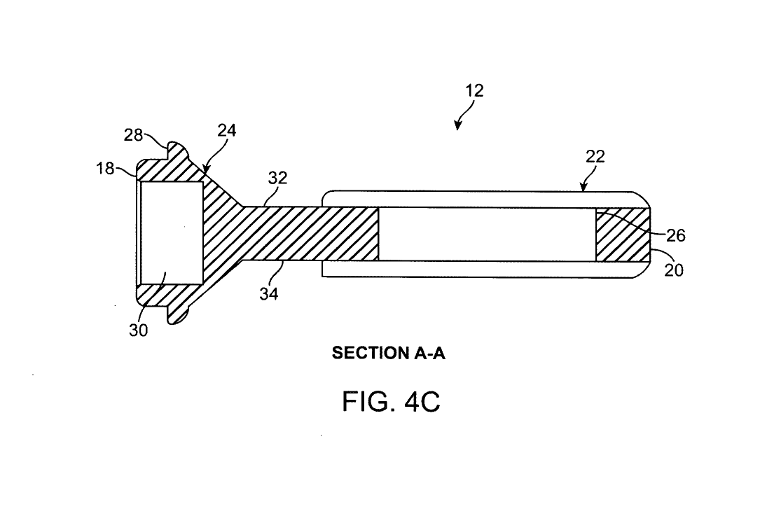

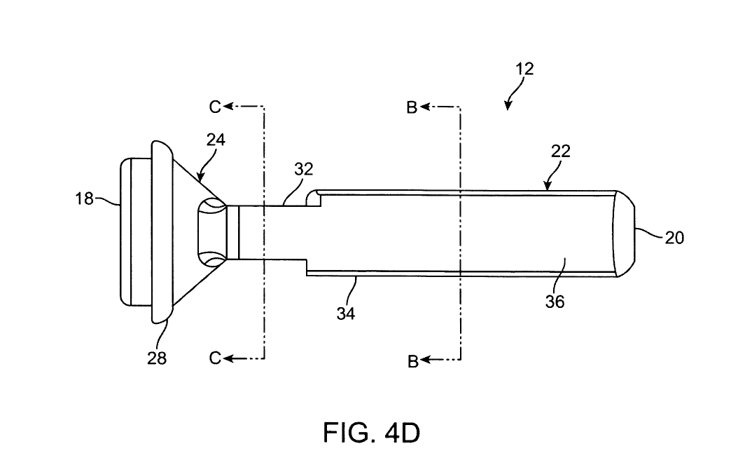

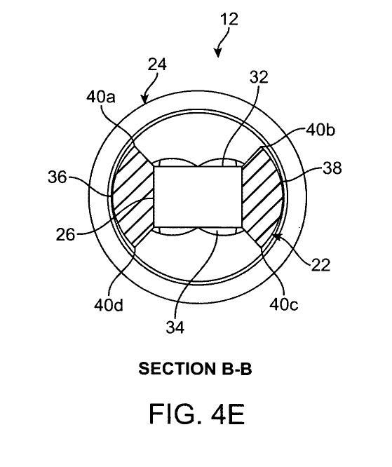

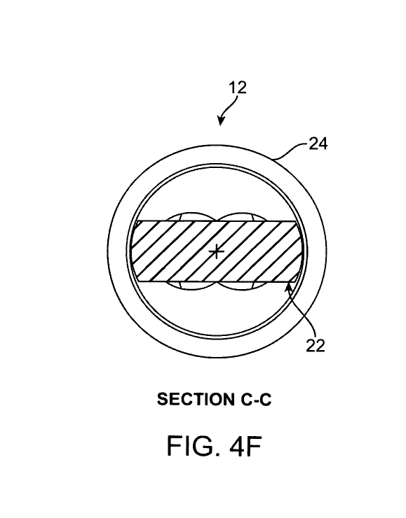

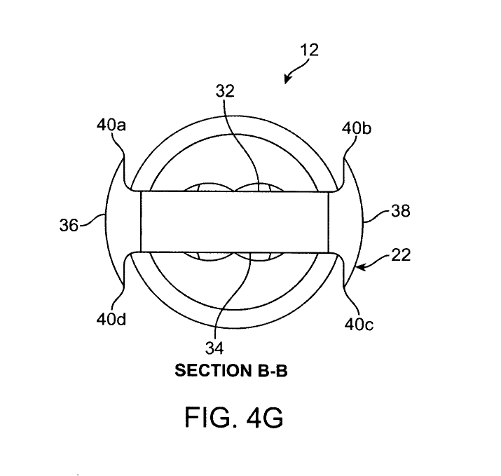

The end bit 12 will now be described with reference to FIGS. 4A, 4B, 4C, 4D and 4F. The end bit 12 has a proximal end 18 and a distal end 20. The end bit 12 includes a cutting head 22 at the distal end 20 connected to a connecting portion 24 at the proximal end 18. With particular reference to FIG. 4C, which illustrates section A-A of FIG. 4B, the connecting portion 26 is substantially circular in cross-section and includes a circumferential flange 28 and an inner bore 30 for connecting to the shaft assembly 14 via insertion of a connecting male member of the shaft assembly 14 into bore 30.

Still referencing FIGS. 4A, 4B, 4C, 4D and 4F, the cutting head 22 of the end bit 12 will now be discussed. The cutting head 22 includes a top surface 32 and a bottom surface 34 interconnected by two sidewalls 36, 38 to form a substantially rectangular block with curved sidewalls; however, in another variation of the invention the cutting head 22 has any functional shape. The sidewalls 36, 38 are curved. In one variation, and both sidewalls 36, 38 trace at least a portion of a circular perimeter. A cavity 26 extends between the top surface 32 and the bottom surface 34. The cavity 26 is elongated in shape and has curved ends as shown in FIG. 4B. The cavity 26 is advantageously configured to provide a location for debris to enter and move through during scraping, thereby, preventing tissue build-up that would otherwise interfere with continued scrapping and cutting. The end bit 12 is made of any suitable material such as surgical steel electopolished and coated with titanium nitride.

With particular reference now to FIG. 4E, the cutting head 22 includes cutting edges or surfaces 40a, 40b, 40c, and 40d. Cutting edges or surfaces 40a and 40d are interconnected by sidewall 36 and cutting edges or surfaces 40b and 40c are interconnected by sidewall 38. Both sidewalls 36 and 38 are curved outwardly to create a convex outer profile on both sides as shown in FIG. 4E. In another variation, both sidewalls 36 and 38 are curved inwardly to create a concave outer profile. Furthermore, the cutting surfaces 40a and 40b extend outwardly from the top surface 32 and are angled at approximately 45 degrees or between approximately 30 and 60 degrees with respect to the top surface 32 and cutting surfaces 40c and 40d extend outwardly from the bottom surface 34 at an angle of approximately 45 degrees or between approximately 30 and 60 degrees with respect to the bottom surface 34. As a result, the angle between cutting surfaces 40a and 40d is approximately 90 degrees and the angle between cutting surfaces 40b and 40c is also approximately 90 degrees. In one variation, at least one of the cutting edges 40a, 40b, 40c and 40d are not diametric but offset slightly from the diameter and not along a diameter. This feature advantageously creates better scrapping action. The distance between the sidewalls 36, 38 is between 5.0 millimeters and 20.0 millimeters. In several variations of the invention, the distance between sidewalls 36, 38 is 7.0 mm, 9.0 mm, 11.0 mm, 13.0 mm, 15.0 mm, and 17.0 mm.

With particular reference now to FIG. 4G, there is shown another variation of the end bit 12 according to the present invention showing a cross-sectional view taken along line B-B of FIG. 4D wherein like numerals are used to describe like parts. The cutting head 22 of FIG. 4G includes cutting edges or surfaces 40a, 40b, 40c, and 40d. Cutting edges or surfaces 40a and 40d are interconnected by sidewall 36 and cutting edges or surfaces 40b and 40c are interconnected by sidewall 38. At least one of the sidewalls 36 and 38 are curved outwardly to create a convex outer profile on at least one of the sides as shown in FIG. 4G. Furthermore, the cutting surfaces 40a and 40b extend outwardly from the top surface 32 and are substantially perpendicular with respect to the top surface 32. Cutting surfaces 40c and 40d extend outwardly from the bottom surface 34 and are substantially perpendicular with respect to the bottom surface 34. As a result, the cutting surfaces 40a and 40d are substantially parallel to each other as are the cutting surfaces 40b and 40c. This variation is another example where the cutting surfaces are not diametric. The distance between the sidewalls 36, 38 is between 5.0 millimeters and 20.0 millimeters. In several variations of the invention, the distance between sidewalls 36, 38 is 7.0 mm, 9.0 mm, 11.0 mm, 13.0 mm, 15.0 mm, and 17.0 mm.

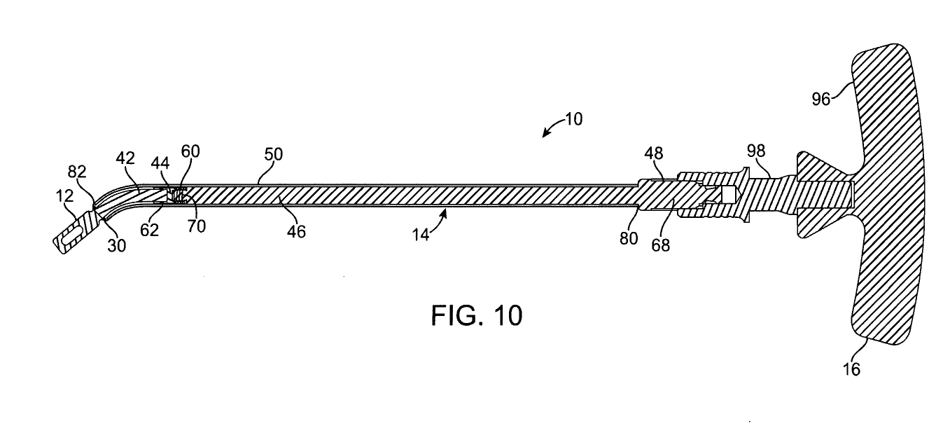

With brief reference to FIG. 10, the shaft assembly 14 of the scraper instrument 10 according to one variation of the invention will now be discussed. The shaft assembly 14 includes a flexible shaft 42, a coupler 44, an inner shaft 46, a ring 48 and an outer shaft 50. The flexible shaft 42 and inner shaft 46 are connected to the coupler 44 and insertable into the outer shaft 50. In another variation, the inner shaft 46 is connected directly to the flexible shaft 42 without the use of a coupler 44. In yet another variation, the shaft assembly 14 comprises an inner shaft 46 and an outer shaft 50 and does not include a flexible shaft 42, coupler 44 and ring 48. Such a variation is suitable for a straight scraper instrument 10 as shown in FIG. 3.

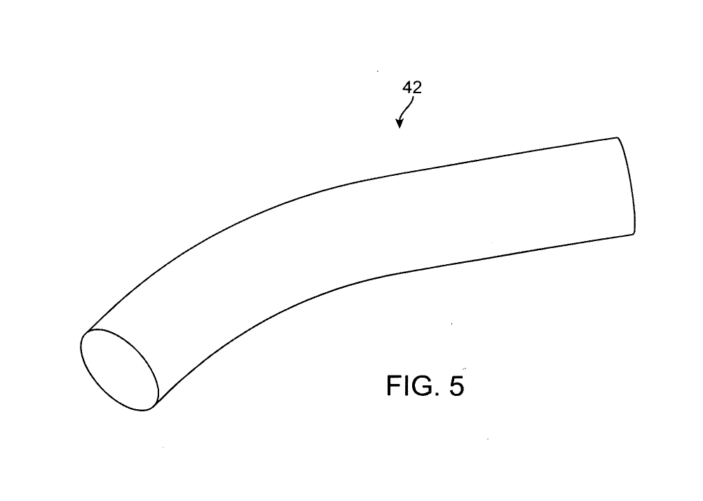

Referring now to FIG. 5, the flexible shaft 42 will now be described. The flexible shaft 42 is generally cylindrical in shape, however, the invention is not so limited and any functional cross-sectional shape is within the scope of the present invention. The flexible shaft 42 is employed to transmit rotational torque applied at the proximal end 18 of the instrument 10 to the distal end 20 of the instrument 10 through an angle located therebetween. In an alternative variation, a system of mechanical linkages is employed to transmit the rotational torque through the bend in the instrument. The flexible shaft 42 includes any connection means necessary such as male or female members at the ends and is made of any kind of suitable material that can withstand the rotational stresses and transfer torque efficiently. For example, the flexible shaft 42 of the present invention can be made from any kind of suitable polymer, stainless steel coiled wire, nitinol or flexible shaft product comprising a slot extending around the shaft such as that manufactured by Nemcomed, Inc. of Hicksville, Ohio and described in U.S. Pat. Nos. 6,447,518 and 6,053,922 incorporated herein by reference in their entireties.

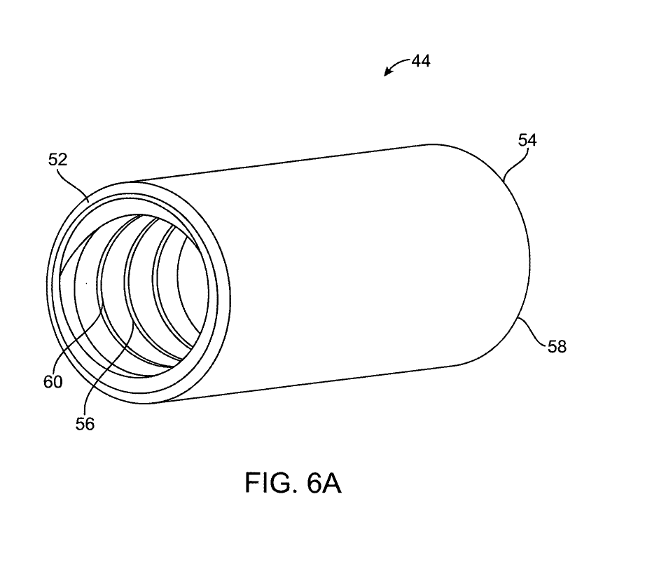

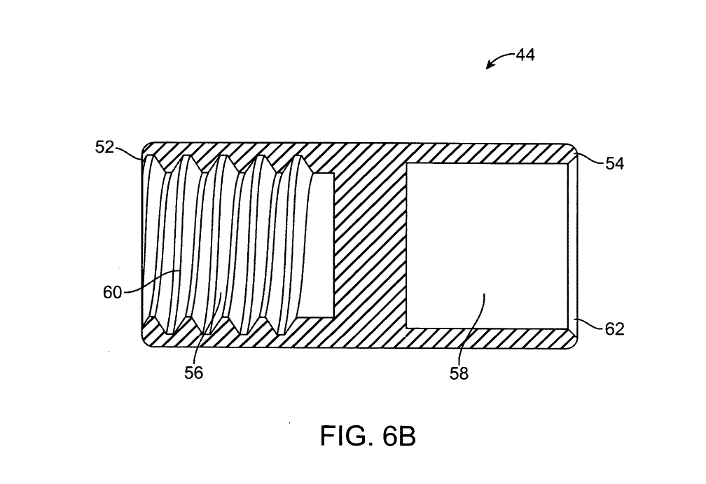

Turning now to FIGS. 6A and 6B and still referencing FIG. 10, the coupler 44 according to one variation of the invention will now be described. As shown in FIGS. 6A and 6B, the coupler 44 is generally cylindrical in shape and includes a proximal end 52 and a distal end 54. The coupler 44 includes an inner shaft receiving portion 56 having an opening at the proximal end 52 and a flexible shaft receiving portion 58 having an opening at the distal end 54 of the coupler 44. The flexible shaft receiving portion 58 includes a bore 62 and the inner shaft receiving portion 56 includes a threaded bore 60 according to one variation of the invention.

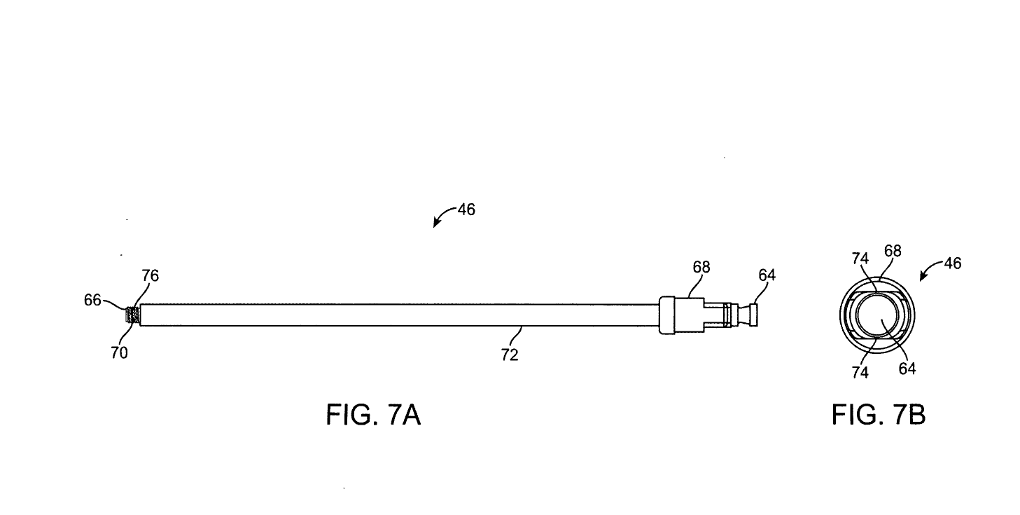

With reference to FIGS. 7A and 7B, the inner shaft 46 will now be described. The inner shaft 46 includes a proximal connector 68 at a proximal end 64 and a distal connector 70 at a distal end 66. The proximal connector 68 and the distal connector 70 are interconnected by a central shaft portion 72. The proximal connector 68 is integrally formed with inner shaft 46 and in another variation it is connected to the inner shaft 46. The proximal connector 68 includes a perimeter that is longer than the circumference of the central shaft portion 72 which helps retain the inner shaft 46 inside the outer shaft 50. The inner shaft 46 in general and the central shaft portion 72 in particular are substantially cylindrical in shape and the inner shaft 46 is made from any suitable material such as stainless steel. The proximal connector 68 is configured to connect with any kind of handle 16 including the handle 16 shown in FIG. 1. The type of handle 16 that the proximal connector 68 is configured to connect to is not limited to a manually operated handle but may also be configured for power operation of the instrument 10. In one variation, the proximal connector 68 is a Hudson male member as shown in FIGS. 7A and 7B. As can be seen in FIG. 7B, the proximal connector 68 includes flat portions 74. In one variation of the invention, there is no proximal connector 68 and the inner shaft 46 is integrally formed with a handle 16. The distal connector 70 is configured to connect with the coupler 44. In one variation, the distal connector 70 includes a threaded male member 76 configured to be inserted into threaded bore 60 of the coupler 44. In another variation of the invention in which no coupler 44 is employed, the distal connector 70 is configured to connect directly with the end bit 12 or flexible shaft 42 and in another variation the distal connector 70 is integrally formed with the end bit 12 or flexible shaft 42. In general, the inner shaft 46 is configured to transmit rotational torque applied at the proximal end 18 of the instrument 10 to the end bit 12 at the distal end 20 and may include any additional structure known to a person of ordinary skill in the art to transmit torque along a length that is straight or includes at least one bend or angle as described below.

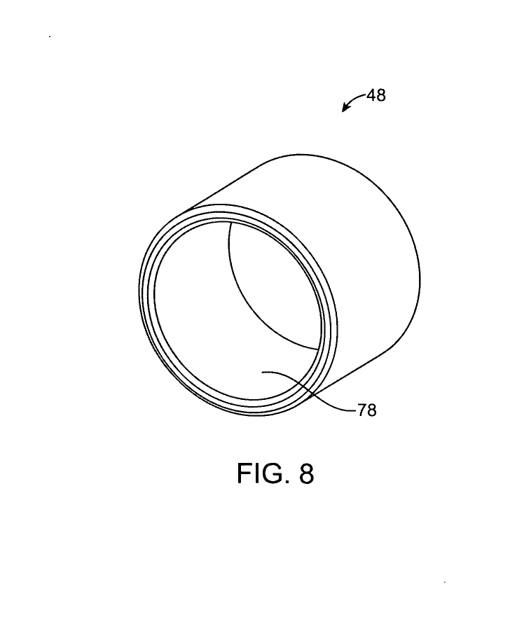

Referring now to FIG. 8 while still referencing FIG. 10, the ring 48 of the shaft assembly 14 will now be described. The ring 48 is substantially cylindrical in shape and has a central bore 78 extending between the proximal end and distal end of the ring 48. The inner diameter of the central bore 78 is sized to be slightly larger than the outer diameter of a portion of the proximal connector 68 of the inner shaft 46. The ring 48 serves as a size indicator for the instrument 10. In one variation, the size of the instrument 10 is defined as the distance between the sidewalls 36, 38 of the end bit 12. The different sizes are indicated with different colored rings 48 thereby color-coding each instrument 10 for ease of identification by the user. In one variation the distance between sidewalls 36, 38 is 7.0 mm, 9.0 mm, 11.0 mm, 13.0 mm, 15.0 mm, and 17.0 mm and the corresponding colors are gold, light blue, magneta, light green, bronze and blue, respectively; however, the invention is not so limited and any size and color combination can be employed.

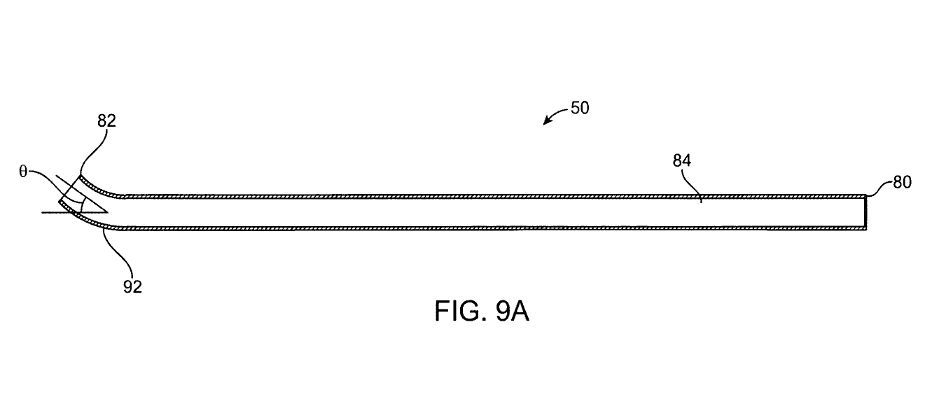

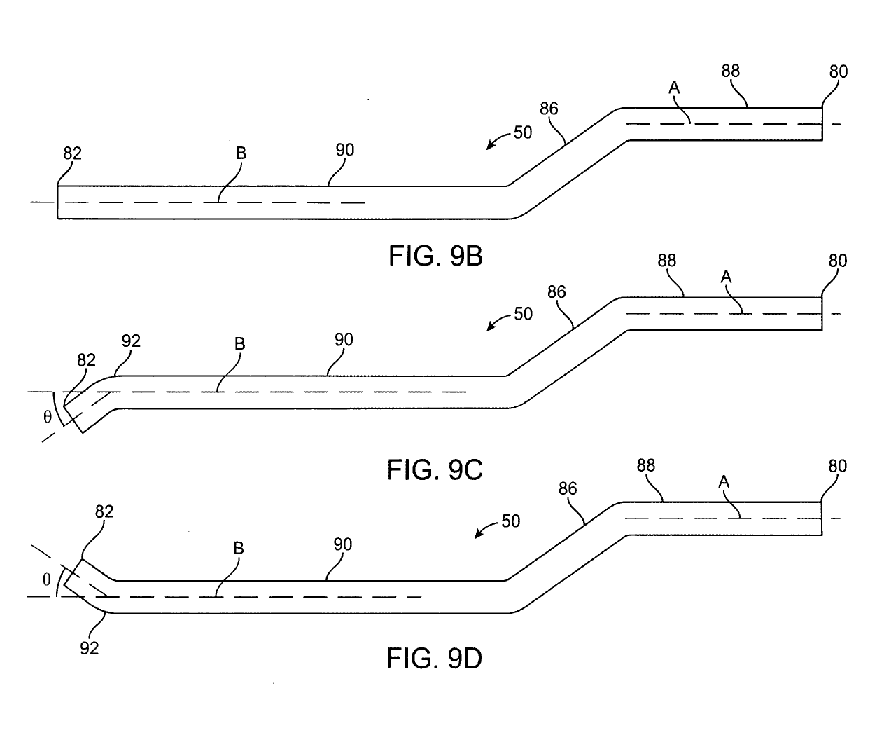

Referring now to FIGS. 9A, 9B, 9C and 9D while still referencing FIG. 10, the outer shaft 50 will now be described. The outer shaft 50 is substantially tubular in shape and has a proximal end 80 and a distal end 82. The outer surface of the shaft 50 includes a diamond knurled portion 94 (shown in FIG. 2) for gripping by the user. The outer shaft 50 includes an outer shaft bore 84 extending between and opening to the proximal end 80 and the distal end 82. The inner diameter of the outer shaft bore 84 is sized slightly larger than the inner shaft 46, the coupler 44 and the flexible shaft 42 in order to receive those elements within the outer shaft bore 84.

FIG. 9A illustrates an outer shaft 50 that includes at least one angled portion 92 near the distal end 82. The angle Θ as shown on FIG. 9A is approximately 40 degrees in one variation of the invention. In another variation, the angle Θ is selected to be any angle between 0 and 90 degrees. In yet another variation of the invention, the angle Θ is adjustable either while the instrument 10 is in use or outside the patient.

Referring to FIGS. 9B, 9C, and 9D, there is shown outer shaft configurations having a bayonet-like shape that includes an angled portion 86 that is midway or closer to the proximal end 80 relative to the distal end 82. At a location along the outer shaft 50 that is between the proximal end 80 and the angled portion 86, the outer shaft 50 defines a first axis A and a first portion 88 of the instrument. A second axis B and second portion 90 of the instrument is defined along the outer shaft 50 at a location between the angled portion 86 and distal end 82. The first axis A is displaced from the second axis B. The first axis A is not aligned with the second axis B. In the variation shown, the first and second axes A and B are shown to be substantially parallel segments 88, 90 that are interconnected by an angled portion 86. The angled portion 86 forming an angle with respect to axis A or B of between approximately 25 degrees and 75 degrees. In a variation that is not shown in the figures, the first axis A is angled with respect to second axis B and not parallel with respect to each other. The outer shaft 50 is configured such that first axis A at the proximal end 80 is sufficiently displaced from second axis B such that the outer shaft 50 between the proximal end 80 and the angled portion 86 does not block or obstruct the space proximally above the outer shaft 50 that is defined between the angled portion 86 and the distal end 82. In another variation, the instrument is configured such that the angled portion 86 is located along the instrument at a location that is resident at or above the outer surface of the patient when in use and inserted into the patient. Therefore, when the instrument 10 is in use and at least a portion of the instrument 10 that is defined between the angled portion 86 and the distal end 82 is inserted into the working channel of a patient in a minimally invasive procedure for example, the first portion 88 of the instrument 10 advantageously does not interfere or obstruct the space above the working channel. The first axis A is displaced from the second axis B by at least the diameter of the outer shaft 50 approximately 5 mm to approximately 25 mm. In another variation, the first axis A is offset from the second axis B by at least half the width or diameter of the first or second portion. FIGS. 9A, 9B, 9C and 9D illustrate variations of the invention that include at least one angled portion. FIGS. 9C and 9D are variations of the invention that include a second angled portion 92 similar to that described with respect to FIG. 9A, with the difference being that in FIG. 9C, the second angled portion 92 is configured such that the distal end 82 points or turns away from the first axis A and, in FIG. 9D, the second angled portion 92 is configured such that the distal end 82 turns back toward the first axis A. The angle Θ variations described with respect to FIG. 9A also apply to the angle Θ variations for FIGS. 9C and 9D and of course, the interior structural components for the variations described in FIGS. 9A, 9B, 9C and 9D are configured as necessary to transmit rotary torque from one end to the other using the structures and methods described herein or as is known to one of ordinary skill in the art.

Turning now to FIG. 10, the assembly of the shaft assembly 14 will now be described. One end of the flexible shaft 42 is inserted into the bore 30 of the end bit 12. Alternatively, the end bit 12 may include a male member that is inserted into a bore formed in the flexible shaft. The other end of the flexible shaft 42 is inserted into the bore 62 of the coupler 44. In an alternative variation in which a coupler 44 is not employed, the other end of the flexible shaft 42 is connected directly to the inner shaft 46. The flexible shaft 42 is secured to the end bit 12 and the coupler 62, or inner shaft 46, using securement means known in the art including but not limited to adhesive, friction fit and weld engagements. The end bit 12, the flexible shaft 42 and the coupler 44, if one is employed, are inserted into distal end 82 of the outer shaft 50. Next, adhesive is applied to the distal connector 70 of the inner shaft 46 and the inner shaft 46 is inserted into the proximal end 80 of the outer shaft 50 and connected to the coupler 44 inside the outer shaft 50. Where the distal connector 70 is a threaded male member 76, it is threaded into the threaded bore 60 of the coupler 44 and locked tight in place. In an alternative variation, the inner shaft 46 is connected directly to the flexible shaft 46 and together inserted into the outer shaft 50; afterwhich, the end bit 12 is connected to the flexible shaft 42 and the proximal connector 68 if separate from the inner shaft 46 is connected to the inner shaft 46. The indicator ring 48 is attached to the proximal end 64 of the inner shaft 46 such that the proximal end 64 is inserted through the bore 78 of the ring 48 and affixed to a visible portion of the proximal connector 68. The inner shaft 46 is retained inside the outer shaft 50 by the circumferential flange 28 of the end bit 12 which prevents the inner shaft 46 from being pulled out as it abuts the distal end 82 of the outer shaft 50. In one variation, the inner shaft 46 is movable with respect to the outer shaft 50. As constructed, the inner shaft 46 and end bit 12 rotate within and with respect to the outer shaft 50. Furthermore, in another variation, the inner shaft 46 is movable via threaded engagement with the outer shaft, for example, such that the outer bit 12 extends along a longitudinal axis of the shaft or generally outwardly from the distal end 82 of the outer shaft 50. This extendible outer bit 12 configuration advantageously permits greater reach and versatility for scraping.

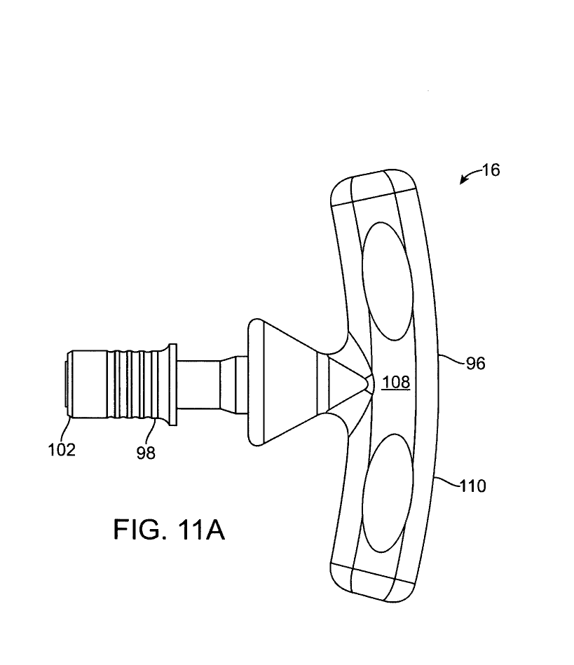

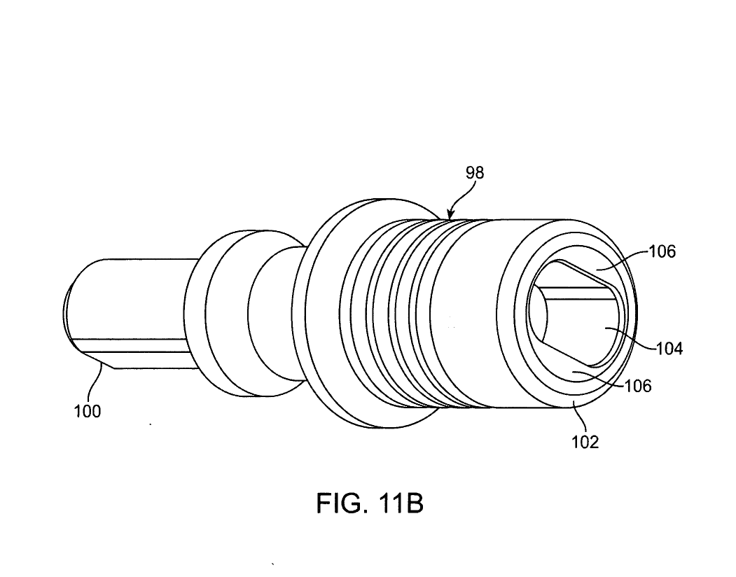

Turning now to FIGS. 11A and 11B, the handle 16 will now be described. In one variation, as described above, the proximal connector 68 of the inner shaft 46 is configured to connect to a power driver for automated operation of the instrument 10. In another variation, the proximal connector 68 is configured to connect to a handle 16. In one case, the handle 16 is removable from the inner shaft 46 and in another variation, the handle 16 is integrally formed with or fixed to the inner shaft 46. The handle 16 includes a T-shaped grasping portion 96 connected to a connecting portion 98. In one variation, the grasping portion 96 and connecting portion 98 are integrally formed. The grasping portion 96 includes a top surface 108 and a bottom surface 110.

One variation of the connecting portion 98 is shown in FIG. 11B. As seen in FIG. 11B, the connecting portion 98 includes a proximal end 100 and a distal end 102. The distal end 102 of the connecting portion 98 is configured to connect to the inner shaft 46 and the proximal end 100 is configured to connect to the grasping portion 96. In one variation, the connecting portion 98 is a quick-connect Hudson adapter configured to connect to the Hudson male member of the inner shaft 46 as shown in FIG. 10. As shown in FIG. 11B, an inner bore 104 opens at the distal end 102 of the connecting portion 98. The inner bore 104 includes substantially flat portions 106. The proximal end 100 is generally connected to the grasping portion 96 and the distal end 102 is connected to the inner shaft 46 and in one variation, the distal end 102 is connected to the inner shaft 46 such that the handle 16 is removably connected to the inner shaft 46.

In another variation, the grasping portion 96 and the distal end 102 of the connecting portion 98 are connected to the inner shaft 46 such that the grasping portion 96 is aligned with the end bit 12. For example, when the flat portions 106 are aligned with flat portions 74 of the inner shaft 46 and, the proximal connector 68 of the inner shaft 46 is inserted into bore 104, the grasping portion 96 is aligned with the end bit 12. In one variation, the alignment of the grasping portion 96 with the end bit 12 is such that a horizontally aligned grasping portion 96 corresponds to a horizontally aligned end bit 12. For example, when aligned, the top surface 32 or bottom surface 34 faces the same direction as the top surface 108 or bottom surface 110 of the grasping portion 96. In another variation, the alignment is such that the grasping portion 96 is rotated 90 degrees with respect to the end bit 12.

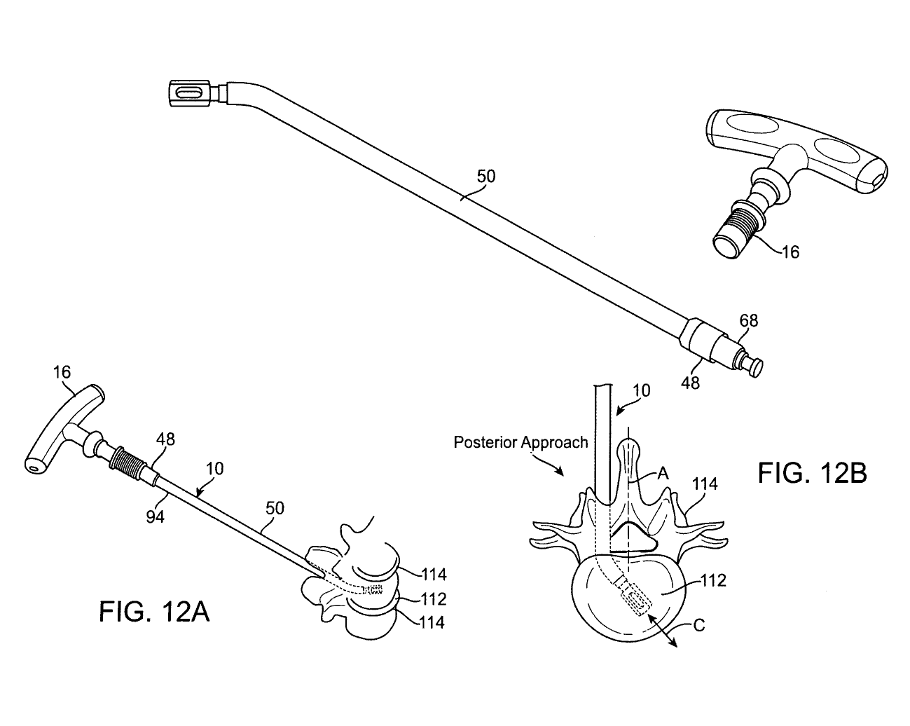

With reference now to FIGS. 12A and 12B, the use of the scraper instrument 10 will now be described. Based on patient anatomy and surgeon preference, an appropriately sized scraper 10 is selected using the indicator ring 48 color code. With or without the handle 16 attached, the instrument 10 is then inserted into the patient, either through a percutaneous cannulated or non-cannulated working channel or open or mini-open operative site in a posterolateral approach that is preferably in the range of an angle of approximately 35 to 90 degrees to an anterior-posterior axis A through the patient in a TLIF procedure or approximately 0 to 65 degrees in a PLIF procedure. Most preferably, the angle is approximately 45 degrees to the anterior-posterior axis through the patient or along the pedicles of the patient. The instrument 10 is passed into a disc space 112 between two adjacent vertebral bodies 114. If a bayonet-style instrument 10, such as any of those described in reference to FIGS. 9B, 9C and 9D, is employed, the instrument 10 does not obstruct the area directly above the working channel or operative site, thereby, facilitating mini-open and percutaneous procedures.

Still referencing FIGS. 12A and 12B, the proximal connector 68 of the inner shaft 46 is connected to a manual handle 16 or power driver. Alignment of the flat portions 74 of the inner shaft 46 and the flat portions 106 of the connecting portion 98 orientates the top and bottom surfaces 32, 34 of the cutting head 22 to substantially face the end plates of the superior and inferior vertebrae as shown in FIGS. 12A and 12B. The outer shaft 50 is held in position by grasping the knurled portion 94. The grasping portion 96 is turned. Rotary torque applied at the grasping portion 96 is transmitted to the end bit 12 through the inner shaft 46, coupler 44, and flexible shaft 42. As a result, the end bit 12 rotates and the cutting edges 40 contact to scrape and remove material at the operative site in the disc space. The end bit 12 can rotate completely around through 360 degrees of rotation. The user turns the handle 16 at user selected angles to reach in and remove desired material and around anatomical features that may need to be avoided or protected. Because of the angled shaft, substantially all of the disc space can be cleared with the instrument. The angle of the instrument with respect to the anterior-posterior axis is changed such that the end bit passes over substantially the entire area of the vertebral endplate. This coverage may be accomplished by also changing the angle of the at least one angled portion in conjunction with or without changing the angle of the instrument with respect to the anterior-posterior axis. In one variation of the instrument 10, the end bit 12 extends and/or retracts from the distal end 82 of the outer shaft 50 and can be moved outwardly and inwardly with respect to the distal end 82 of the outer shaft 50 in a direction shown by the arrows C in FIG. 12B. Removed debris passes through the cavity 26 of the end bit 12 to permit unimpeded cutting and removal. The endplates of the vertebral bodies are prepared by scraping away desired material and by getting the surfaces to bleed and provide a vascular source for in growth and fusion. The disc space is then ready to receive an implant.

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

We claim:

1. A scraper instrument, comprising:
a shaft assembly having at least one angled portion comprising:
a outer shaft with a central bore extending from a proximal end to a distal end;
an inner shaft disposed inside the central bore of the outer shaft such that the inner shaft is permitted to rotate with respect to the outer shaft; the inner shaft having a proximal end and a distal end;
a flexible shaft having a proximal end and a distal end; the proximal end of the flexible shaft connected to the distal end of the inner shaft wherein the flexible shaft is located at the angled portion;
an end bit having a proximal end and a distal end; the proximal end of the end bit connected to the distal end of the flexible shaft; the end bit having a cutting head at the distal end; the cutting head having at least one cutting surface; and
wherein rotational torque applied to the proximal end of the inner shaft is transmitted to the flexible shaft at the angled portion and to the end bit to rotate the cutting head.

2. The scraper instrument of claim 1 wherein the cutting head includes a top surface and a bottom surface interconnected by two sidewalls, forming four cutting surfaces; wherein first and fourth cutting surfaces are interconnected by a first sidewall and second and third cutting surfaces are interconnected by a second sidewall.

3. The scraper instrument of claim 2 wherein at least one of the sidewalls is curved outwardly for a substantially convex outer profile.

4. The scraper instrument of claim 2 wherein at least one of the sidewalls is curved inwardly for a substantially concave outer profile.

5. The scraper instrument of claim 2 wherein at least one of the cutting surfaces are angled with respect to the top surface.

6. The scraper instrument of claim 2 wherein at least one of the cutting surfaces are substantially perpendicular to the top surface.

7. The scraper instrument of claim 2 wherein at least one of the four cutting surfaces is not diametric.

8. The scraper instrument of claim 2 further including a cavity extending from the top surface to the bottom surface.

9. The scraper instrument of claim 1 wherein the at least one angled portion defines two parallel axes offset from each other to form a bayonet-like configuration for the instrument.

10. The scraper instrument of claim 1 wherein the angle of the at least one angled portion is adjustable.

11. The scraper instrument of claim 1 further including a coupler having a proximal end and a distal end; the coupler being connected between the inner shaft and the end bit.

12. A scraper instrument, comprising p1 a rotatable inner shaft;
a rotatable end bit having a cutting head; the end bit being connected to the inner shaft; and
an outer shaft connected to the inner shaft such that the inner shaft is permitted to move relative to the outer shaft.

13. The scraper instrument of claim 12 wherein the cutting head includes a top surface and a bottom surface interconnected by two sidewalls, forming four cutting surfaces; wherein first and fourth cutting surfaces are interconnected by a first sidewall and second and third cutting surfaces are interconnected by a second sidewall.

14. The scraper instrument of claim 13 wherein at least one of the sidewalls is curved.

15. The scraper instrument of claim 13 wherein at least one of the cutting surfaces is angled with respect to the top surface.

16. The scraper instrument of claim 13 wherein at least one of the cutting surfaces are substantially perpendicular to the top surface.

17. A scraper instrument of claim 12 wherein the end bit is extendable and retractable with respect to the distal end of the outer shaft.

18. A scraper instrument of claim 12 further including at least one angled portion and a flexible shaft; the flexible shaft being interconnected between the inner shaft and the end bit and configured to transmit rotational torque that is applied to the proximal end of the inner shaft through the angled portion.

19. The scraper instrument of claim 18 wherein the at least one angled portion is proximate to the distal end of the instrument.

20. The scraper instrument of claim 18 wherein the at least one angled portion is located between the proximal end and distal end of the outer shaft with a first portion defining a first axis located between the proximal end and the angled portion and a second portion defining a second axis located between the angled portion and the distal end.

21. The scraper instrument of claim 20 wherein the at least one angled portion is located along the instrument such that it is resident at or above the surface of the patient while in use and inserted into the patient.

22. The scraper instrument of claim 20 wherein the first axis is substantially parallel and offset from the second axis.

23. The scraper instrument of claim 22 wherein the first axis is offset from the second axis by at least half of the width of the first or second portion.

24. The scraper instrument of claim 21 further including a second angled portion proximate the distal end.

25. The scraper instrument of claim 21 wherein the angle of the at least one angled portion is adjustable.

26. A method comprising:
providing a scraper instrument having a rotatable end bit with at least one cutting surface;
creating an working channel to the disc space of a patient;
inserting the scraper instrument through the working channel to the disc space in a posterolateral approach that is in the range of an angle of approximately 0 to 90 degrees to an anterior-posterior axis of a patient's spine;
rotating the end bit to remove tissue in the disc space;
changing the angle of the instrument to pass the end bit over substantially the entire area of a vertebral endplate.

27. The method of claim 26 further including the step of extending or retracting the end bit from the distal end of the instrument.

28. The method of claim 26 wherein the step of providing a scraper instrument further includes providing a scraper instrument that has at least one angled portion.

29. The method of claim 26 wherein the step of inserting the scraper instrument includes inserting the scraper instrument in a posterolateral approach that is in the range of an angle of approximately 35-90 degrees to the anterior-posterior axis.
